(19) United States
(12) Patent Application Publication
(10) Pub. No.: US 2012/0143082 A1
(43) Pub. Date: Jun. 7, 2012

(54) TISSUE SAMPLE ANALYSIS

(76) Inventors: Ioan Notingher, Nottingham (GB); Hywel Williams, Nottingham (GB); William Perkins, Nottingham (GB); Sandeep Varma, Nottingham (GB)

(21) Appl. No.: 13/320,094

(22) PCT Filed: May 13, 2010
(86) PCT No.: PCT/GB2010/050786

§ 371 Date: Feb. 23, 2012

(30) Foreign Application Priority Data
May 13, 2009 (GB) 0908204.1

(51) Int. Cl. A61B 10/02; G01N 21/65

(52) U.S. Cl. 600/562; 435/34

(57) ABSTRACT

A new method of Raman micro spectroscopy for the detection and imaging of Basal Cell Carcinoma (BCC) comprises the application of a multivariate supervised statistical classification model to distinguish between dermis, epidermis and BCC. The resulting Raman images provide a tool for the automated and objective evaluation of a tissue sample.

The present invention relates to tissue sample analysis, and in particular to a new method of Raman spectroscopy for detection and imaging of Basal Cell Carcinoma (BCC), together with associated multivariate statistical classification model; tissue analysis apparatus and computer program products.

1. Introduction

Skin cancer is a growing source of concern, not only for being the most common of all types of cancers, and the less reported, but also due to its alarming high-increasing incidence rate. Each year, there are more new cases of skin cancer than the combined incidence of cancers of breast, prostate, lung and colon. (Cancer Research UK (a), 2008). Only in UK and USA more than 100,000 and 1,000,000 cases, respectively, are diagnosed annually (Cancer Research UK (a), 2008; American Cancer Society (a), 2008).

About 80% of skin cancer cases worldwide are Basal Cell Carcinomas (BCGs). This type of skin cancer belongs to the keratinocyte or non-melanoma family, and begins in the lowest layer of the epidermis (the basal cell layer). It usually occurs in areas exposed to the sun, such as the head or the neck (American Cancer Society (b), 2008). For large, rare or recurrent BCCs, for those growing into the surrounding skin tissue or in critical areas, e.g. the high risk zone of the face (nasolabial folds, eyelids and periauricular areas) Mohs Micrographic Surgery (MMS) is the most suitable treatment (Cancer Research UK (b), 2007).

MMS was first developed by Frederick Mohs and maximises the evaluation of the surgical margin by pathologic observation of the histological slides during surgery. Sequential layers of tissue are removed until the lesion is clear of BCC. If the pathologic evaluation indicates tumour persistence, accurate location is recorded and further tissue removal is performed by the surgeon. This procedure ensures high cure rates and allows maximal conservation of healthy tissue which can be particularly important on areas such as the face.

It is widely accepted that Mohs Micrographic Surgery (MMS) is the most effective current method for removal of aggressive BCC in terms of compromise between maximum conservation of healthy skin and minimum recurrence rates. (McGovern, 1999; Smeets et al., 2004; Telfer et al., 1999).

While 5-years recurrence rates for BCC treated by MMS are 1.4% for primary tumours and 4% for recurrent tumours (Leibovitch et al., 2005), for standard excision, this rate reaches 3.2% to 10% for primary tumours, and more than 17% for recurrent BCCs (Rowe et al., 1989). It has also been reported that at shorter follow-up periods (18 months), MMS was considerably more advantageous than surgical excision especially for recurrent tumours (0% versus 3%) (Smeets et al., 2004).

However, in many cases, traditional methods such as surgical excision, cryosurgery, radiotherapy, curettage and electrodessication rather than MMS are applied for high risk BCC removal, despite its lower effectiveness, based solely on availability and cost considerations. (Bialy et al, 2004), (Essers et al, 2006), (Bath-Hextall et al, 2004). Indeed, focusing on UK as an example of current worldwide situation, it has been reported the fact that there are less MMS centres and specialist surgeons than the number recommended by the medical community according to clinical needs (NICE report, UK, 2006). The main reason for the inequity of service provision is the need of time consuming procedures to obtain and evaluate tissue sections during MMS, as well as specialized staff including expert histopathologists for diagnosis and trained technicians for frozen sections manipulation.

In addition, several studies have reported that even the gold-standard of histopathology has inter-observer differences^{[36]}. In a study on 48 samples evaluated by 20 pathologists, overall sensitivity was 87% (range, 55%-100%) and specificity 94% (range, 83%-100%)^{[37]}. Other study on 592 histopathology slides using two pathologists, inter-observer agreement was found only in 93% of cases^{[38].} However, the real values for effectiveness in BCC diagnosis during MMS in terms of sensitivity and specificity may be lower because most MMS surgeons are not trained histopathologists.

Therefore, an automated reliable low-cost method for BCC detection and imaging in MMS excised skin sections as an alternative to current histopathology tissue evaluation, would allow a wider use of MMS according to clinical need. This would be a significant advance in the management of BCCs.

According to a first aspect of the present invention, there is provided a method of analysing a tissue sample comprising
obtaining micro Raman spectra from one or more locations across the sample;
applying a multivariate supervised statistical classification model to predict the class of each spectrum as dermis, epidermis or basal cell carcinoma (BCC).

Optionally, said method further comprises creating a quantitative Raman spectroscopic image of the sample based on the classification from the model.

Optionally, said image represents any regions of dermis, epidermis and BCC that are present in the sample with different colours for each class of region.

Optionally, said step of applying a multivariate supervised statistical classification model comprises a linear discriminant analysis (LDA) model.

Optionally, an unsupervised k-means clustering is performed on the obtained micro Raman spectra, and then peak ratios of the centroids of each of those clusters are introduced as input to the LDA model and classified as dermis, epidermis or BCC.

Alternatively, the LDA model is applied directly to the micro Raman spectra to classify each unknown spectra as dermis, epidermis or BCC.

Optionally, neighbouring spectra are binned and averaged together with the sample spectrum to reduce variability due to tissue heterogeneity.

Optionally, the model uses selected Raman bands chosen to reflect the biochemical differences between BCC and healthy skin regions.

Optionally said selected Raman bands comprise vibrations specific to collagen 1, said bands from the sample being compared with the model to differentiate dermis from BCC.

Optionally, said vibrations specific to collagen 1 comprise vibrations corresponding to at least one of O—P—O phosphodiester and PO₂.

Optionally said selected Raman bands comprise vibrations specific to DNA, said bands from the sample being compared with the model to differentiate epidermis from BCC.

Optionally, said vibrations specific to DNA comprise vibrations corresponding to at least one of C—C vibrations in protein backbones and Amide III vibrations in protein.

Optionally, said selected Raman bands are represented as the ratios of peak intensities at the selected Raman bands with the intensity of a reference band that shows low differences between dermis, epidermis and BCC classes.

Optionally, said reference band corresponds to the ring breathing of phenylalanine.

Optionally, said ratios are introduced as the input parameters in two consecutive linear discriminant analyses.

Optionally, a first linear discriminant analysis is performed to discriminate BCC and epidermis from dermis; and a second linear discriminant analysis is performed to discriminate BCC from epidermis.

According to a second aspect of the present invention, there is provided a multivariate supervised statistical classification model to predict the class of each spectrum as dermis, epidermis or basal cell carcinoma (BCC).

Optionally, said model comprises data derived from the mean spectra of a plurality of tissue samples.

Optionally, the model uses selected Raman bands chosen to reflect the biochemical differences between BCC and healthy skin regions.

Optionally said selected Raman bands comprise vibrations specific to collagen 1, said bands from the sample being compared with the model to differentiate dermis from BCC.

Optionally, said vibrations specific to collagen 1 comprise vibrations corresponding to at least one of O—P—O phosphodiester and PO₂.

Optionally said selected Raman bands comprise vibrations specific to DNA, said bands from the sample being compared with the model to differentiate epidermis from BCC.

Optionally, said vibrations specific to DNA comprise vibrations corresponding to at least one of C-C vibrations in protein backbones and Amide III vibrations in protein.

Optionally, said selected Raman bands are represented as the ratios of peak intensities at the selected Raman bands with the intensity of a reference band that shows low differences between dermis, epidermis and BCC classes.

Optionally, said reference band corresponds to the ring breathing of phenylalanine.

According to a third aspect of the present invention, there is provided apparatus for analysing a tissue sample comprising:
a stage for receiving a tissue sample;
a Raman micro-spectrometer; and
a multivariate supervised statistical classification model arranged to be applied to the micro Raman spectra of the tissue sample obtained from the Raman micro-spectrometer, to predict the class of each spectrum as dermis, epidermis or basal cell carcinoma (BCC).

Optionally, said apparatus comprises means to display a quantitative Raman spectroscopic image of the sample based on the classification from the model.

Optionally, said image represents any regions of dermis, epidermis and BCC that are present in the sample with different colours for each class of region.

Optionally, said multivariate supervised statistical classification model comprises a linear discriminant analysis (LDA) model.

Optionally, said apparatus comprises means for performing an unsupervised k-means clustering on the obtained micro Raman spectra, and to introduce peak ratios of the centroids of each of those clusters as classified dermis, epidermis or BCC inputs to the LDA model.

Alternatively, said apparatus comprises means for applying the LDA model directly to the micro Raman spectra to classify each unknown spectra as dermis, epidermis or BCC.

Optionally, said apparatus comprises means for binning and averaging together neighbouring spectra with the sample spectrum to reduce variability due to tissue heterogeneity.

Optionally, said model is arranged to use selected Raman bands chosen to reflect the biochemical differences between BCC and healthy skin regions.

Optionally said selected Raman bands comprise vibrations specific to collagen 1, said bands from the sample being compared with the model to differentiate dermis from BCC.

Optionally, said vibrations specific to collagen 1 comprise vibrations corresponding to at least one of O—P—O phosphodiester and PO₂.

Optionally said selected Raman bands comprise vibrations specific to DNA, said bands from the sample being compared with the model to differentiate epidermis from BCC.

Optionally, said vibrations specific to DNA comprise vibrations corresponding to at least one of C—C vibrations in protein backbones and Amide III vibrations in protein.

Optionally, said selected Raman bands are represented as the ratios of peak intensities at the selected Raman bands with the intensity of a reference band that shows low differences between dermis, epidermis and BCC classes.

Optionally, said reference band corresponds to the ring breathing of phenylalanine.

Optionally, said ratios are introduced as the input parameters in two consecutive linear discriminant analyses.

Optionally, a first linear discriminant analysis is performed to discriminate BCC and epidermis from dermis; and a second linear discriminant analysis is performed to discriminate BCC from epidermis.

According to a fourth aspect of the present invention, there is provided a computer program product carrying instructions for the performance of the first aspect.

According to a fifth aspect of the present invention, there is provided a computer program product carrying instructions for the embodiment of the second aspect.

According to a sixth aspect of the present invention, there is provided a computer program product carrying instructions for control of the apparatus of the third aspect.

The computer program product of any of the fourth to sixth aspects may comprise computer readable code embodied on a computer readable recording medium. The computer readable recording medium may be any device storing or suitable for storing data in a form that can be read by a computer system, such as for example read-only memory (ROM), random-access memory (RAM), CD-ROMs, magnetic tapes, floppy disks, optical data storage devices, and carrier waves (such as data transmission through packet switched networks such as the Internet, or other networks). The computer readable recording medium can also be distributed over network coupled computer systems so that the computer readable code is stored and executed in a distributed fashion. Also, the development of functional programs, codes, and code segments for accomplishing the present invention will be apparent to those skilled in the art to which the present disclosure pertains.

According to a seventh aspect of the present invention, there is provided a method for the surgical removal of a BCC tumor, comprising removing a first tissue sample from a patient; analysing the first tissue sample according to the method of the first aspect; and removing a subsequent tissue sample from the patient based on the or any detection of BCC in the first tissue sample.

The present invention will now be described, by way of example only, with reference to the accompanying drawings, in which:

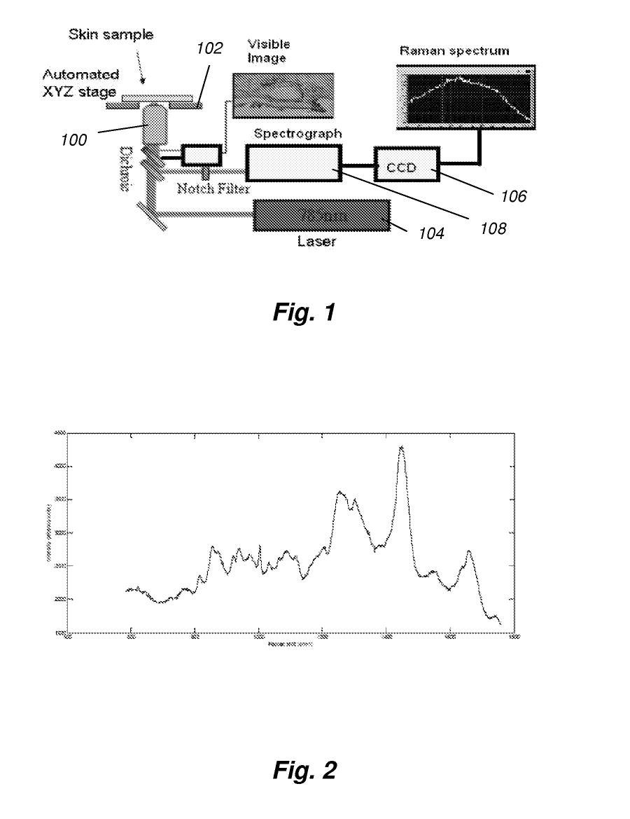

FIG. 1 illustrates a schematic description of a Raman micro-spectrometer according to an embodiment of the present disclosure;

FIG. 2 illustrates a typical Raman spectrum of skin;

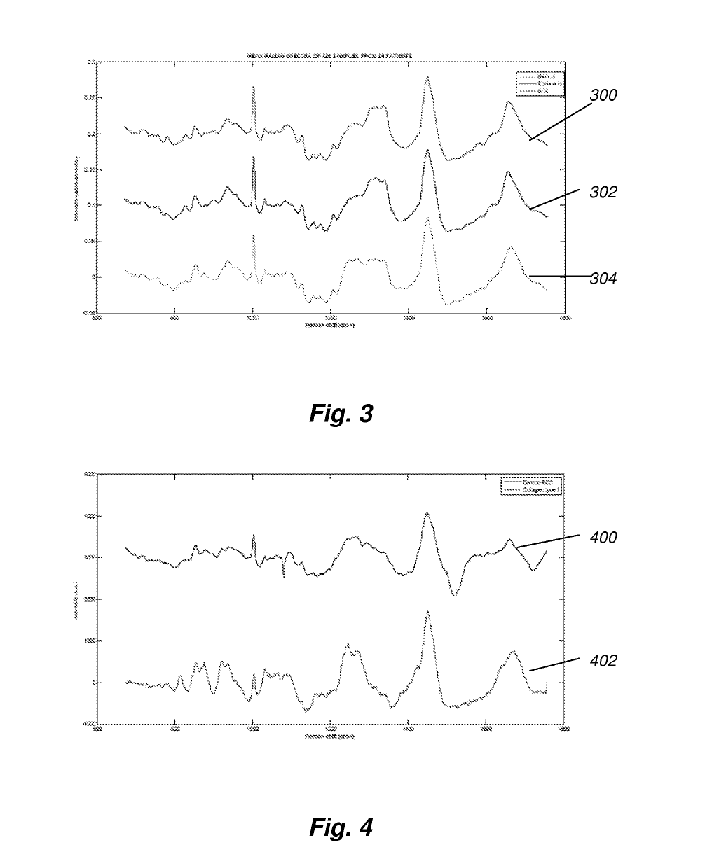

FIG. 3 illustrates mean Raman spectra of 329 tissue specimens (127 BCCs, 92 epidermis and 110 dermis), from 20 patients used to construct a multivariate classification model;

FIG. 4 illustrates a comparison between the Raman spectra of “Dermis minus BCC” and the Raman spectra of Collagen type I;

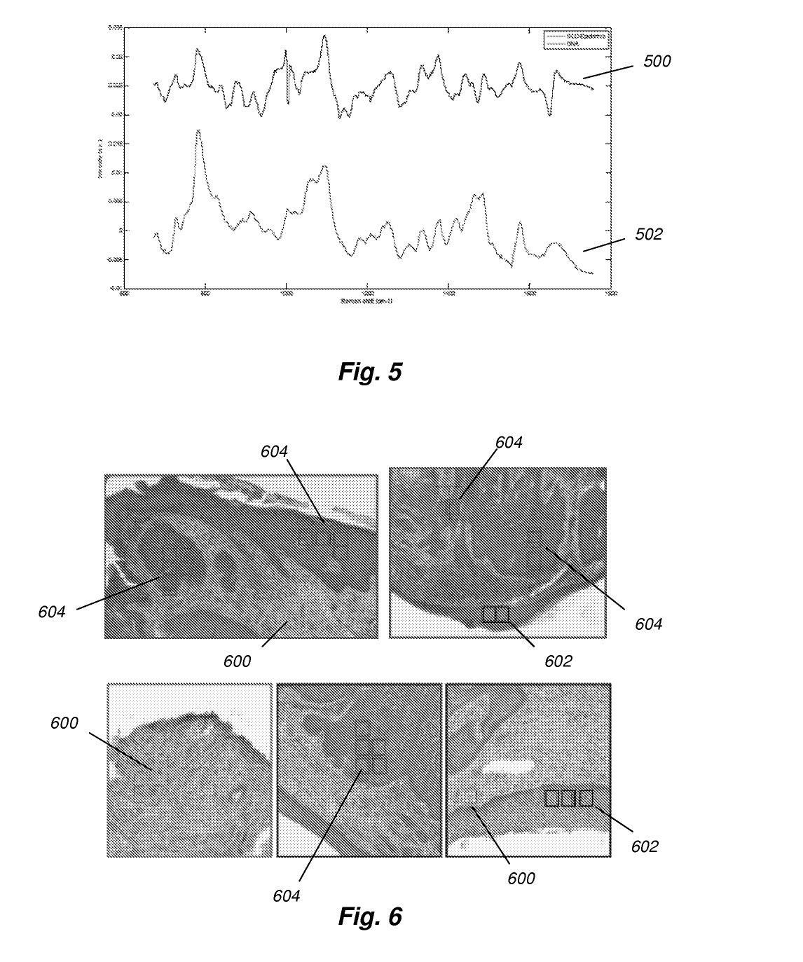

FIG. 5 illustrates a comparison between the Raman spectra of “BCC minus epidermis” and the Raman spectra of DNA;

FIG. 6 illustrates several H&E images of skin tissue, showing typical measured regions of dermis, epidermis and BCC;

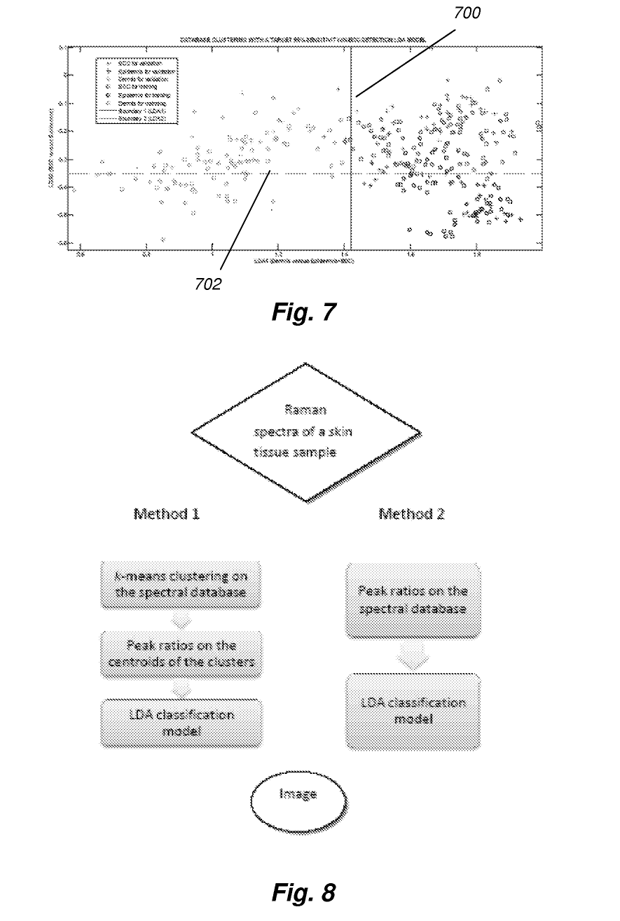

FIG. 7 illustrates a typical result of a cross-validation procedure, where data from a spectral database has been classified into 3 groups, red for BCC, blue for epidermis and green for dermis;

FIG. 8 illustrates two alternative supervised procedures for building 2-D biochemical images of skin tissue sections;

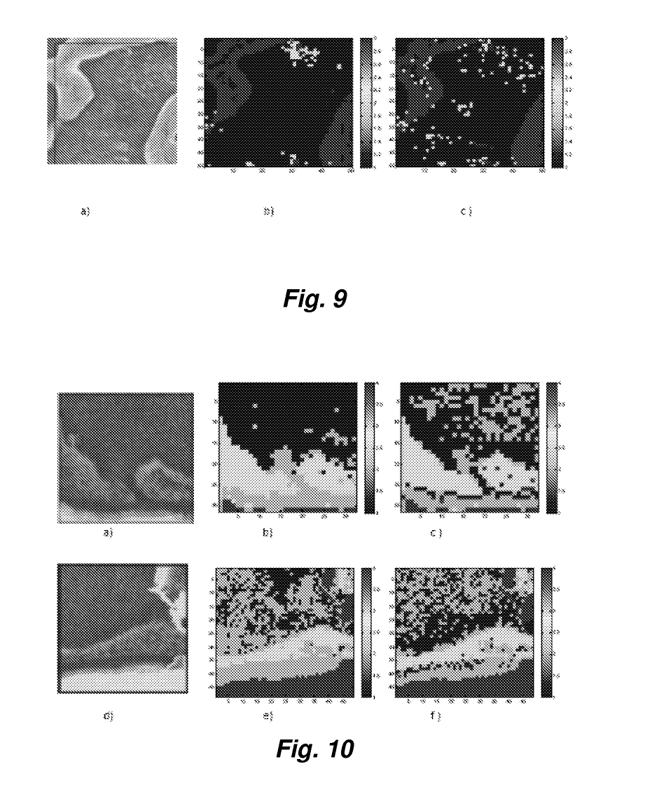

FIG. 9 illustrates a comparison among Raman images produced with the two supervised methods of FIG. 8 and corresponding histopathological H&E image of an MMS excised skin tissue sample of 500 μm by 500 μm containing BCC;

FIG. 10 illustrates a comparison among Raman images produced with the two supervised methods of FIG. 8 and corresponding histopathological H&E images of two MMS excised skin tissue samples of 480 μm by 465 μm and 480 μm by 480 μm, respectively, containing nodular BCC;

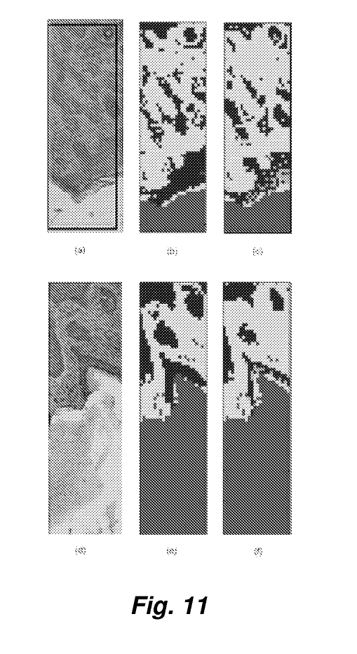

FIG. 11 illustrates a comparison among Raman images produced with the two supervised methods of FIG. 8 and corresponding histopathological H&E images of two MMS excised skin tissue samples of 240 μm by 720 μm and 240 μm by 840 μm, respectively, containing morphoeic BCC; and

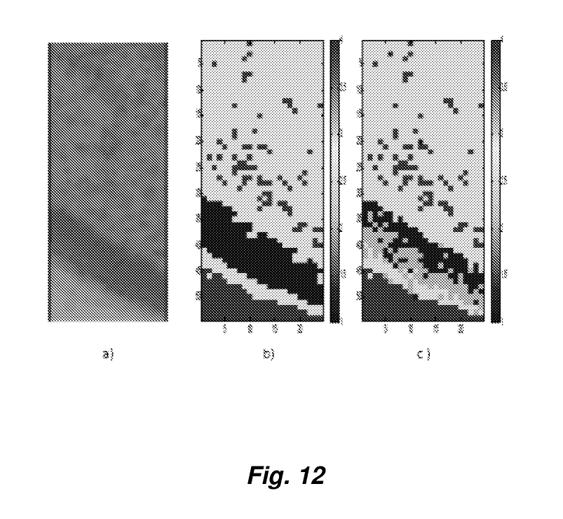

FIG. 12 illustrates a comparison among Raman images produced with the two supervised methods of FIG. 8 and corresponding histopathological H&E image of an MMS excised skin tissue samples of 240 μm by 540 μm clear of BCC.

The technique proposed herein for creating images of skin tissue excised during MMS uses Raman Micro-Spectroscopy (RMS). The spectra produced from this technique may be referred to as “micro Raman spectra”. In RMS, the Raman signal of different micrometric regions within a sample is collected to produce an image based on the biochemical composition of the sample. Therefore, Raman spectra are ‘chemical fingerprints’ of the constituents of the sample and are based on the Raman effect.

For the last two decades, RMS has been recognised as a powerful optical technique for biomedical applications (Manoharan et al., 1996). Compared to fluorescent spectroscopy, vibrational spectra of biomolecules are characterised by molecule specific narrow peaks, which are sensitive to molecular structure, conformation and interactions. This high chemical specificity constitutes a major advantage of RMS, being able to detect slight chemical changes in biological samples (Kumar et al, 2007). RMS achieves diffraction-limited lateral resolution in the micrometer range, which makes it an appropriate tool to imaging cells and tissues (Krafft et al, 2006).

RMS is a suitable technique for cancer diagnosis because of its high sensitivity to molecular and structural changes associated with cancer, such as an increased nucleus-to-cytoplasm ratio, disordered chromatin, higher metabolic activity, and changes in lipid and protein levels (Keller et al, 2006). Many studies have been reported attempting to discriminate between cancer and healthy cells for different tissues in vitro and in vivo, in animal and human tissue. Examples of exhaustive animal tissue cancer discrimination are the researches done by Amharref et al on rat brain tissue samples (Amharref et al, 2007) and Bakker et al for in vivo discrimination of rat dysplastic tissue (Bakker et al, 2000). The potential of RMS for detection and diagnosis of human cancers, both in vivo and in vitro, has been demonstrated for a large number of cancer types, including skin (Gniadecka et al, 1997), (Gaspers et al, 1998), (Hata et al, 2000), (Nijssen et al, 2002), (Lieber et al, 2008), breast (Haka et al., 2005), oesophagus (Kendall et al., 2003), lung (Huang et al., 2003), cervix (Murali Krishna et al, 2006) and prostate (Crow et al., 2003).

Early studies on skin using RMS presented Raman Spectroscopy as a useful tool in dermatological diagnosis, comparing Raman spectra from normal, healthy human stratum corneum with other skin tissues, such as callus tissue or hyperkeratotic psoriatic plaques (Edwards et al, 1995). The capability of Raman Spectroscopy to detect biochemical alterations in skin tissue caused by BCC was first demonstrated by Gniadecka et al. 1997a. Several protein and lipid alterations characteristics of BCC tissue, such as the alterations of the amide bands, attributed to the conformational changes of proteins (essentially, changes in collagen), were reported (Gniadecka et al., 1997.a & 1997.b). Further experiments showed 97% sensitivity and 98% specificity on BCC detection were realized using Principal Component Analysis (PCA) for dimension reduction along with a neural network classifier for spectral clustering (Gniadecka et al, 2004). A more recent work also demonstrated the ability of RMS combined with fibre optics for skin tumour in vivo diagnosis (Lieber et al, 2008).

However, apart from tumour detection, MMS requires imaging of BCC regions in tissue blocks and sections. Quantitative Raman spectroscopic images can be built by representing the intensity of a certain spectral peak, score or weight obtained with a multivariate spectral analysis method for each individual location in the 2-D region where Raman spectra were acquired. Raman spectroscopic measurements do not require sample preparation, e.g. dying the tissue, they are free of variations due to changes in the molecular composition and structure of the sample which may be caused by preparation protocols. Consequently, RMS is an objective and quantitative method that can be used continuously with the same level of accuracy, making it ideally suitable for automatic implementation and biochemical imaging. Since RMS does not rely on light absorption, tissue thickness and water tissue these have little effect on the measurements. Therefore, this technique could be used both on tissue sections and excised tissue blocks.

Many studies have applied this technique to tumour discrimination, to create Raman maps of tissue sections containing cancerous cells. Images of brain, gastrointestinal (GI) tract (Shetty et al, 2006), lymph nodes (Romeo et Diem, 2005), lung (Krafft et al, 2008) and skin (Nijssen et al, 2002) cancer have been created. These studies employed unsupervised methods for imaging, such as the intensities of the scores of a selected number of principal components or k-means clustering. However, these unsupervised methods for creating Raman images have an important disadvantage when applied to detection and imaging of tumours: for building a specific image of a tissue sample only information present in this particular tissue is used. Therefore, images obtained by these methods show require additional expert information to produce a medical diagnosis. Therefore, supervised methods are more advantageous because information from a large number of samples and patients are used be used to provide diagnosis of new skin sections.

In this disclosure, a supervised classification method has been developed to investigate the ability of RMS to detect and image BCC in skin tissue excised during MMS and skin surgery. A spectral database using 329 tissue specimens from 20 randomly chosen patients was developed. The spectra were divided into three classes, BCC, dermis or epidermis, according to histopathology diagnosis. Once the classification accuracy was established, the model was applied on tissue specimens obtained from new patients for imaging of tumour regions.

2. Methods and Materials.

2.1 Skin Tissue Samples

Skin tissue sections were obtained from the Nottingham University Hospitals NHS Trust. Consent was obtained from the patients and ethical approval was granted from Nottingham Research Ethics Committee. Tissue sections were cut from blocks removed during MMS and standard BCC excision into 20 μm sections for RMS investigations. After the RMS measurements, the analysed sections were stained using conventional Haematoxylin and Eosin (H&E) staining and diagnosis was given by a consultant histopathologist. An adjacent tissue section was also obtained and H&E stained to provide guidance for the selection of tumour regions to be analysed by RMS.

2.2 Raman Spectroscopy

FIG. 1 shows a schematic description of an apparatus suitable for carrying out the methods of the present disclosure. It will be appreciated that the precise arrangement shown is for the purposes of illustration only and that other equivalent set-ups could be used. The apparatus may comprise an inverted microscope 100 (1×71 Olympus) equipped with an automated XYZ translation stage 102 (for example, as available from Prior Ltd), 785 nm laser 104 (Toptica) 50 mW at sample, deep-depletion back-illuminated CCD detector 106 and spectrograph 108 (both from Andor Ltd). Microscopy cameras (2.1 Infinity and MF cool Jenoptik) were used for recording images of the tissue sections. Other components of the apparatus include a dichroic beam splitter and a notch filter, as shown in FIG. 1. The visible image and Raman spectrum from a skin sample are also shown. The wavenumber axis was calibrated using Raman standard samples (ASTM E 1840), such as naphthalene and 1,4 bis(2-methylstyryl benzene) (Sigma). The wavenumber accuracy was found to be +/−0.5 cm⁻¹.

2.3 Building the RMS Database for BCC Discrimination

2.3.1 Data Acquisition:

First, an adjacent Haematoxylin & Eosin (H&E) stained skin section was placed on the microscope and the regions of interest (BCC, epidermis or dermis), identified. Its corresponding unstained skin section, which had been deposited on a MgF₂ window, was placed on the microscope. Helped by the H&E section, the regions for taking measurements were selected and coordinates of position recorded. After RMS measurements, the section was returned to the pathology lab, H&E stained and then placed on the Raman microscope for retrospective acquisition of images to be used for diagnosis by a consultant histopathologist and classified into three classes: BCC, epidermis or dermis. The precision of retrospective location was determined to be less than 5 μm based on two marks engraved on each slide.

To account for tissue heterogeneity, each Raman spectrum in the model represented the average of 100 spectra measured at 5 μm intervals over a 50 μm by 50 μm region. The integration time for each position was 1 second. A total of 329 measurements from 20 patients have been recorded: 127 BCCs (nodular and morphemic), 92 epidermis and 110 dermis. A typical spectrum used for building the model is shown in FIG. 2, which shows a typical Raman spectrum of the skin acquired at a single position in 1 second and an average spectrum over a 50 μm×50 μm without preprocessing. The intensity (arbitrary units) is plotted against the Raman shift (cm⁻¹).

2.3.2 Data Analysis:

Prior to analysis, the contribution of the microscope objective was subtracted. All spectra were baseline corrected using a 6^{th} order polynomial and normalised to zero mean and unity standard deviation. Finally data were smoothed with Savitsky-Golay algorithm (5 points, 2nd order polynomial).

This disclosure proposes to perform data analysis using Linear Discriminant Analysis (LDA). The LDA model was built using the area of several selected Raman peaks which showed highest contrast in the computed difference average spectra for each class. The boundaries of the LDA model were set for 95% target sensitivity and the prediction sensitivity and specificity were calculated using 70%-30% split cross-validation (leave-one-out cross-validation was also used for comparison with previous studies).

2.4 Spectral Imaging of BCC

After the LDA model was built, the ability of RMS to detect and image BCC was tested on a new set of 6 skin sections obtained from 3 patients (no samples from these patients were included into the LDA model). Raman spectra of a selected region were acquired at 5 μm intervals with 2 second integration time.

Spectra were binned over 10 or 15 μm to account for tissue heterogeneity, i.e., each new spectra was the average of the 4 or 9 adjacent spectra. Thus, spatial resolution of biochemical images achieved was 10 or 15 μm, respectively. Then, the proposed LDA model was applied to predict the class of each spectrum as BCC, epidermis or dermis. An image was then constructed based on the LDA model classification.

3. Results and Discussion.

3.1 Spectral Database.

Mean spectra of the 329 measured tissue specimens (127 BCCs, 92 epidermis and 110 dermis) from 20 patients used to construct the multivariate classification model are presented in FIG. 3, showing mean Raman spectra of 329 tissue specimens (127 BCCs, 92 epidermis and 110 dermis), from 20 patients used to construct the model. Note that spectra have been vertically shifted an arbitrary quantity to avoid overlapping. This figure shows that there exist spectral differences between BCC (graph 300) and epidermis (graph 302) or dermis (graph 304), in agreement with previous reported works (Gniadecka et al, in 1997), (Gaspers et al, 1998), (Nijssen, 2002). The main differences between dermis and BCC are due mainly to the presence of collagen I in dermis and not in BCC, as inferred from the computed spectrum difference (shown in FIG. 4—this figure shows a comparison between the Raman spectra of “Dermis minus BCC” (graph 400) and the Raman spectra of Collagen type I (graph 402). It reveals a higher presence of Collagen I in dermis than in BCC).

In addition, the main differences between BCC and epidermis can be explained by the higher presence of DNA in the tumour tissue, as shown in FIG. 5. This figure shows a comparison between the Raman spectra of “BCC minus epidermis” (graph 500) and the Raman spectra of DNA (graph 502). It shows a higher percentage of DNA in BCC than in epidermis.

Higher presence of DNA is caused by the higher density of cells present in the tumour, as can be seen in FIG. 6, where several H&E images of typical measured skin tissue regions of 50 μm×50 μm are presented, being represented as empty squares in typical dermis regions 600, epidermis regions 602 and BCC regions 604. H&E staining images show DNA in dark brown and dermis in pale orange. Therefore, regions with higher DNA will be darker, as it is the case of cancerous areas. In opposition, zones with higher collagen will present a paler colour, as it is the case of dermis.

3.2 LDA Classification Model.

Based on the comparison among the mean Raman spectra of the three classes forming the spectral database shown in FIGS. 3-5, six peak intensities have been chosen as ‘fingerprints’ to classify the spectra. The selection criterion was to maximize the differences among classes, therefore including the main peaks of the DNA and the collagen I. The ratios of the peak intensities chosen were:

$${r}_{1} = \frac{{I}_{788 {cm}^{- 1}}}{{I}_{1003 {cm}^{- 1}}} , {r}_{2} = \frac{{I}_{850 {cm}^{- 1}}}{{I}_{1003 {cm}^{- 1}}} , {r}_{3} = \frac{{I}_{950 {cm}^{- 1}}}{{I}_{1003 {cm}^{- 1}}} , \\ {r}_{4} = \frac{{I}_{1093 {cm}^{- 1}}}{{I}_{1003 {cm}^{- 1}}} , {r}_{5} = \frac{{I}_{1312 {cm}^{- 1}}}{{I}_{1268 {cm}^{- 1}}} ,$$

These Raman bands can be assigned to specific vibrations in DNA and collagen type I. The 788 cm⁻¹ and 1093 cm⁻¹ correspond to the O—P—O phosphodiester respectively to PO₂ vibrations in DNA. The bands at 850 cm⁻¹ and 950 cm⁻¹ are associated to the C—C vibrations in protein backbones while the area between 1200-1350 cm^{□1} has been assigned to Amide III vibrations in protein (Manoharan et al., 1996). The intensity of the 1003 cm⁻¹ corresponding to the ring breathing of phenylalanine has been chosen as denominator of the ratio because it showed low differences between classes.

Introducing the ratios of the peak intensities, i.e., r₁, r₂, r₃, r₄ and r₅, as input parameters in two consecutive Linear Discriminant Analysis, it has been possible to discriminate, first, BCC and epidermis from dermis and second, BCC from epidermis. The order of the LDAs takes into account the easiest discrimination among dermis and the other two classes elucidated from FIG. 3. The model showed that RMS is able to discriminate nodular and morphoeic BCC from healthy tissue with 90±9% sensitivity and 85±9% specificity in a 70%-30% split cross-validation algorithm (95% target sensitivity). The final value achieved for sensitivities and specifities and its correspondent calculated error correspond to the mean of randomly chosen partitions of our dataset into training and validation for the model.

A typical result of a cross-validation procedure is shown in FIG. 7, where the 329 data from the spectral database have been classified into 3 groups, red for BCC, blue for epidermis and green for dermis. 70% of the data were used for training the model, and are represented in the figure as empty circles. The other 30% were used for validation of the model, and their symbol in FIG. 7 is a cross. The employed algorithm consisted of two consecutive LDAs. In the first place, dermis is separated from the other two classes, BCC and Epidermis, that are considered one only group, by LDA (LDA1). Then, BCC is separated from epidermis by a new LDA (LDA2). A first boundary line 700 and a second boundary line 702 represent the 95% target sensitivity discrimination lines of LDA1 and LDA2 respectively.

FIG. 7 shows that there is a significant clustering of the spectra into three groups corresponding to BCC, epidermis and dermis. However, BCC and epidermis clusters overlap, as it is expected due to the great similarities between their mean spectra (see FIGS. 3-5).

From results shown in FIGS. 3-5, mean Raman spectra of dermis and BCC present clear differences in some of the selected peaks employed by our model, such as those of r₅, i.e., the main collagen peaks. Thus, it could be expected hardly any of the dermis to be misclassified as BCC and vice versa. However, FIG. 7 shows that there are several dermis spectra located in the middle of the BCC cluster. Inspection of these spectra showed that they were more similar to the BCC mean spectrum of FIGS. 3-5 than to the mean Raman spectra of the dermis presented in the same figures. Correlation with the H&E images indicated that spectra indicate regions of inflamed dermis, which had a higher amount of cell nuclei than normal dermis. The variability in Raman spectra of dermis depending on its distance to the tumour has already been reported (Nijssen et al, 2002).

3.3 Raman Spectral Imaging.

Once the LDA model was built, it was applied to create 2-D biochemical images of tissue sections using two different procedures. The first method used unsupervised k-means to group the Raman spectra according to spectral differences. To ensure discrimination between BCC and epidermis, 11 classes were required. As k-means clustering is an unsupervised method, the presence of any skin-irrelevant element or alteration in the sample may be detected and classified first as a new class. After splitting the spectra into 11 clusters, the peak ratios of the centroids of each of those clusters were introduced as input in our LDA model and classified as BCC, epidermis or dermis.

The second method consisted on applying directly the LDA model to the individual Raman spectra measured at each location of the analysed tissue to classify each unknown spectra as BCC, epidermis or dermis. The schemes of both procedures are presented in FIG. 8.

FIG. 9 shows a comparison among Raman images produced with the two supervised methods proposed in this paper (in FIGS. 9b and 9c) and its corresponding histopathology examined H&E image (in FIG. 9a) of an MMS excised skin tissue sample of 500 μm by 500 μm containing BCC. In the Raman maps, brown means dermis, green epidermis and blue 5CC. For the H&E image, BCC corresponds to the dark brown region while pale orange corresponds to dermis. There are also paler regions of inflamed dermis in the dark brown stain. The tissue sample does not contain glass or epidermis. FIG. 9b is produced by the first method (k-means clustering and LDA model) while FIG. 9c is produced by the second method direct LDA model).

To reduce variability due to tissue heterogeneity, neighbouring spectra were binned. Therefore, each pixel in FIG. 9 (and the subsequent FIGS. 11 and 12) corresponds to the average of 4 recorded spectra and in FIG. 10 to the average of 9 spectra. In addition, each spectrum was smoothed with Savitsky-Golay algorithm (5 points, 2nd order polynomial). Note that averaging of neighbour spectra to build the Raman maps results in a scaling difference between pixels in the H&E image and those belonging to the Raman images. Results using classification methods 1 and 2 for imaging are presented in FIGS. 9 to 12. These figures show excellent agreement with the gold standard of histopathology.

FIG. 9 shows the ability of the RMS models to image tumour regions in sections containing only BCC and dermis, as the spectra differences are higher. BCC is correctly detected and both dermis and cancer accurately located within the tissue. Only a few regions of inflamed dermis are being misclassified as epidermis, perhaps due to a lower amount of DNA at those locations. Note that each pixel in the Raman maps is the average of 4 spectra in the H&E image, which introduces a scaling difference. FIG. 9c shows that applying the LDA model to individual spectra leads to some misclassification of epidermis as dermis.

Secondly, the technique was used to detect nodular BCC in sections containing all three regions included in the model, BCC, epidermis and dermis. This is illustrated in FIG. 10, which shows examples of nodular BCC, showing a comparison among Raman images produced with the two supervised methods proposed herein (FIGS. 10{b,e} and {c,f} and its corresponding histopathology examined H&E image (FIG. 10a) of two MMS excised skin tissue samples of 480 μm by 465 μm and 480 μm by 480 μm, respectively, containing nodular BCC. In the Raman maps, yellow means dermis, light blue epidermis, dark blue BCC and brown means glass. For the H&E image, the following colour code is employed: yellow corresponds to the glass; the dark brown region located within the tissue is BCC, while the darker horizontal line parallel to the glass is epidermis. The paler orange regions present are dermis, and the external orange line in direct contact with the glass corresponds to stratum corneum (epidermis). FIGS. 10b and e are produced by the first method (k-means clustering and LDA model) while FIGS. 10c and f are produced by the second method (direct LDA model).

The correlation of the spectral images with the H&E images is good, the dermis is correctly identified despite the presence of a large number of cells. Again the k-means-LDA model has a better accuracy in classification of epidermis and less misclassification of BCC as epidermis.

Excellent agreement with H&E staining images was also obtained with morphoeic BCC.

FIG. 11 shows a comparison among Raman images produced with the two supervised methods proposed herein (in FIGS. 11{b,e} and {c,f}) and its corresponding histopathology examined H&E image (in FIGS. 11a and d), of a MMS excised skin tissue sample of 240 μm by 720 μm and 240 μm by 840 μm, respectively, containing morphoeic BCC. In the Raman maps, yellow means dermis, light blue epidermis, dark blue BCC and brown means glass. For the H&E image, the following colour code is employed: yellow corresponds to the glass; the dark brown region located within the tissue is BCC while if it is in contact with the glass is epidermis. The paler region is dermis, that becomes darker if it is highly inflamed. FIGS. 11b and e are produced by the first method (k-means clustering and LDA model) while FIGS. 11c and f are produced by the second method (direct IDA model).

BCC regions as small as 30-40 μm were detected. However, in this case, the k-means-LDA method showed higher number of epidermis misclassification than direct application of LDA model to each individual spectra.

From a clinical point of view, it is crucial that both classification methods were able to detect with high accuracy the presence of nodular and morphoeic BCC within the tissue sections, as well as the dermis regions. Nevertheless, due to the overlap of BCC and epidermis clusters shown in FIG. 7 some of the epidermis were misclassified as BCC (see FIG. 11, b-c and e-f). However, this misclassification of epidermis as BCC has a less clinical significance, because if a region at the edge of the tissue is predicted as BCC but shows no BCC regions within the dermis, it is likely that it is misclassified epidermis. Also, areas located deep into the sample been predicted as epidermis are more likely to be BCCs or very highly inflamed dermis than epidermis, unless they belong to hair follicles.

Finally, the technique was applied to skin tissue sections excised during MMS which were declared a clear of BCC, as shown in FIG. 12.

FIG. 12 shows the comparison among Raman images produced with the two supervised methods proposed herein (FIGS. 12b and 12c) and its corresponding histopathology examined H&E image (FIG. 12a), of an MMS excised skin tissue sample of 240 μm by 540 μm clear of BCC. In the Raman maps, yellow means dermis, light blue epidermis, dark blue BCC and brown means glass. For the H&E image, the following colour code is employed: yellow corresponds to the glass; the dark brown is epidermis and the paler region is dermis. FIG. 12b is produced by the first method (k-means clustering and LDA model) while FIG. 12c is produced by the second method (direct LDA model).

Both methods where able to detect dermis with high accuracy and no BCC regions were predicted within the dermis. Some epidermis is misclassified as BCC, particularly buy the k-means-LDA method, due to the higher spectral similarities between BCC and epidermis.

Therefore, due to the current lower specificity, we envisage that initially RMS may be used to image all tissue layers removed during MMS and only the sections declared clear of BCC or where BCC is detected at the edge of the tissue section, will be evaluated by the surgeon using frozen sections to check that no BCC was missed. As the prediction accuracy and specificity increases in time, the RMS could be used on its own eliminating the need of pathology observation. These potential changes in surgery practice will improve the MMS efficiency, allowing all BCC patients to benefit from the best treatment available.

4. Conclusion

We have shown that RMS using supervised classification models can be used for detection and imaging of BCC within MMS skin tissue sections, as a feasible alternative to histopathology.

The LDA-model was developed using 329 Raman spectra from 20 patients, including 127 BCC, 92 epidermis and 110 dermis. BCC was discriminated from healthy tissue with 90±9% sensitivity and 85±9% specificity in a 70%-30% split cross-validation algorithm (95% target sensitivity). Once the model was developed, it was applied to build 2D biochemical images of unknown skin tissue samples excised during MMS. The images were obtained by using two supervised methods. The first one applies k-means clustering to the whole spectral database, and then the peak ratios of the spectra of each centroid are introduced into the LDA classification model and labelled as dermis, epidermis or BCC. The second method directly applies the LDA model over the peak ratios of the whole spectral database.

This analysis represents a new line to previous works in RMS imaging, where unsupervised classification methods had been chosen to create the Raman maps. Images produced by both methods reveal the presence/absence of tumour without intervention from histopathologists and determined its location within the sample.

This result may have an important implication on the management of BCC: it may be used to detect in situ the presence/absence of cancer in a sample, allowing the surgeon to continue with the surgery process while the tumour presence remains clear in the excised sections, without the need to send all the intermediate sections to the histopathology lab. Nevertheless, at the current stage, the technique cannot be used alone without the surveillance of a surgeon, who will state the final diagnosis, but it constitutes an important advance in BCC imaging and a promising improvement in MMS feasibility. Its direct application during MMS to the excised skin tissue sections will reduce the number of samples to be processed in the histopathology lab, minimizing time and costs of MMS and, thus, broadening its use, which is nowadays very restricted due to the two previously mentioned factors.

Future work on improving the model with a larger amount of samples from different patients and introducing new classes such as inflamed dermis, hair follicles or sebaceous glands may be carried out. Also a reduction in the time acquisition of the Raman spectra from the skin tissue sections may be useful.

Basal Cell Carcinoma (BCC) constitutes about 80% of diagnosed skin cancers. For aggressive BCCs, Mohs Micrographic Surgery (MMS) is considered the most suitable treatment. Its main disadvantage is the need of frozen section preparation and histopathology examination for all excised tissues, a non-automated, time-consuming technique.

Raman Micro-Spectroscopy (RMS) can be used as an alternative to histopathology during MMS to produce an automated and objective method for evaluation of skin tissues. RMS and multivariate supervised statistical classification models were developed for detection and imaging of BCC regions. The model was built using selected Raman bands and Linear Discriminant Analysis (LDA) on 329 Raman spectra measured from skin specimens from 20 patients. The selected Raman bands reflected the biochemical differences between BCC and healthy skin regions (mainly corresponding to DNA and collagen type I). BCC was discriminated from healthy tissue with 90±9% sensitivity and 85±9% specificity in a 70%-30% split cross-validation algorithm (95% target sensitivity). This multivariate model was then applied on tissue sections obtained from new patients with the aim of imaging tumour regions. The RMS image showed excellent correlation with the golden-standard of histopathology images, BCC being detected in all sections.

This study demonstrates the potential of RMS for an automated objective method for tumour evaluation during MMS. The replacement of current histopathology during MMS by a ‘generalization’ of the proposed technique may improve the feasibility and efficacy of MMS, leading to a wider use according to clinical need.

Various improvements and modifications may be made to the above, without departing from the scope of the invention.

### REFERENCES

The following references have been mentioned above, and are incorporated herein by reference in their entirety, to the maximum extent permissible.

[1] Cancer Research UK (a). Revised: May 6, 2009. Accessed: May 2009. Available at URL address: http://info.cancerresearchuk.org/cancerstats/types/skin/

[2] American Cancer Society (a). Cancer Facts & Figures 2008. Revised: 2008. Accessed: May 2009. Available at URL address: http://www.cancer.org/downloads/STT/2008CAFFfinalsecured.pdf

[3] American Cancer Society (b). What Are Basal and Squamous Cell Skin Cancers? Revised: Jul. 30, 2008. Accessed Nov. 15, 2008. Available at URL address: http://www.cancer.org/docroot/CRI/content/CRI_{—}2_{—}2_{—}1x_What_Is_Nonmelanoma_Skin_Cancer_{—}51.asp?sitearea
[4] Cancer Research UK. Skin cancer (b). Treating skin cancer. Surgery. Updated May 15, 2007. Accessed Nov. 15, 2008. Available at URL address: http://www.cancerhelp.org.uk/help/defaultasp?page=4362#mohs

[5] T W. McGovern, “Mohs Surgery: The Informed View”, Arch. Dermatol. 135, 1255-59 (1999).

[6] N. Smeets, G. Krekels, J. Ostertag, B. Essers, C. Dirksen, F. Nieman, H. Neumann, “Surgical excision Mohs' micrographic surgery for basal□cell carcinoma of the face: randomised controlled trial”, The Lancet 364 (9447), 1766-1772 (2004).

[7] N. R. Telfer, G. B. Colver, W. Bowers, “Guidelines for the management of basal cell carcinoma”, Br. J. Dermatol., 141 (3), 415-423 (1999).

[8] Leibovitch I, Huilgol S C, Selva D, Richards S, Paver R, “Basal cell carcinoma treated with Mohs surgery in Australia 11. Outcome at 5-year follow-up”, J Am Acad Dermatol 53, 452-457(2005).

[9] R. Rowe, R. Carroll, C. Day, “Long-Term Recurrence Rates in Previously Untreated (Primary) Basal Cell Carcinoma: Implications for Patient Follow-Up”, J. Dermatol. Surg. Oncol., 15 (3), 315328 (1989).

[10] T. L. Bialy, J. Whalen, E. Veledar, D. Lafreniere, J. Spiro, T. Chartier, S. C. Chen “Mohs Micrographic Surgery vs Traditional Surgical Excision: A Cost Comparison Analysis”, Arch. Dermatol. 140, 736-742 (2004).

[11] B. A. B. Essers, C. D. Dirksen, F. H. M. Nieman, N. W. J. Smeets, G. A. M. Krekels, M. H. Prins, H. A. M. Neumann, “Cost-effectiveness of Mohs Micrographic Surgery vs Surgical Excision for Basal Cell Carcinoma of the Face” Arch. Dermatol. 142, 187-194 (2006).

[12] F. Bath-Hextall, J. Bong, W. Perkins, H. Williams, “Interventions for basal cell carcinoma of the skin: systematic review”, B. M. J., 329, 705-9 (2004)

[13] National Institute for Health snd Clinical Excellence 2006; “Improving outcomes for people with skin tumours including melanoma”. Issued: February 2006. Accessed: May 2009. Available at URL address: http://www.nice.org.uk/guidance/CSGSTIM

[14] R. Manoharan, Y. Wang, M. S. Feld, “Histochemical analysis of biological tissues using Raman spectroscopy”, Spectrochim Acta Part A 52, 215-49 (1996).

[15] K. K. Kumar, A. Anand, M. V. P. Chowdary, K. Thakur, J. Kurien, C. M. Krishna, S. Mathew “Discrimination of normal and malignant stomach mucosal tissues by Raman spectroscopy: a pilot study”. Vib. Spectrosc. 44: 382-387 (2007).

[16] C. Krafft, S. B. Sobottka, G. Schackert, R. Salzer, “ Raman and infrared spectroscopic mapping of human primary intracranial tumors: a comparative study”, J. Raman Spectrosc. 37, 367-375(2006).

[17] Keller M., Kanter, E. M, Mahadevan-Jansen, A., “Raman Spectroscopy for Cancer Diagnosis”, Spectrosc. 21(11), 33 (2006).

[18] N. Amharref, A. Beljebbar, S. Dukic, L. Venteo, L. Schneider, M. Pluot, M. Manfait, “Discriminating healthy from tumor and necrosis tissue in rat brain tissue samples by Raman spectral”, Biochim Biophys Acta 1768 , 2605-2615 (2007).

[19] T. C. Bakker Schut, M. J. Witjes, H. J. Sterenborg, O. C. Speelman, J. L. Roodenburg, E. T. Marple, H. A. Bruining, G. J. Puppels, “In vivo detection of dysplastic tissue by Raman spectroscopy”, Anal Chem. 72 6010-6018 (2000).

[20] Gniadecka M, Wulf H C, Nielsen O F, Christensen D H, Hercogova J: “Distinctive molecular abnormalities in benign and malignant skin lesions: Studies by Raman spectroscopy”. Photochem. Photobiol. 66, 418-423 (1997.a).

[21] Gaspers P J, Lucassen G W, Wolthuis R, Bruining H A, Puppels G J, “In Vitro and In Vivo Raman Spectroscopy of Human Skin”Biospectrosc. 4 (S5), S31-S39 (1998).

[22] Hata T R, Scholz T A, Ermakov I V, McClane R W, Khachik F, Gellermann W, Pershing L K. “Non-invasive Raman spectroscopic detection of carotenoids in human skin”. J. Invest. Dermatol.; 115(3) 441-448(2000).

[23] A. Nijssen, T. C. B. Schut, F. Heule, P. J. Gaspers, D. P. Hayes, M. H. Neumann, G. J. Puppels, “Discriminating Basal Cell Carcinoma from its Surrounding Tissue by Raman Spectroscopy”, J. Invest. Dermatol., 119 , 64-69 (2002).

[24] Lieber C A, Majumder S K, Ellis D J, Billheimer D D, Mahadevan-Jansen A. “In Vivo Nonmelanoma Skin Cancer Diagnosis Using Raman Microspectroscopy”, Laser Surg Med 40 461-467 (2008).

[25] A. S. Haka, K. E. Shafer□Peltier, M. Fitzmaurice, J. Crowe, R. R. Dasari and M. S. Feld, “Diagnosing breast cancer by using Raman spectroscopy”, Proc. Natl. Acad. Sci. USA, 102, 12371-12376 (2005).

[26] C. Kendall, N. Shepherd, B. Warren, K. Geboes, H. Barr, N. Stone. ‘Raman spectroscopy a potential tool for the objective identification and classification of neoplasia in Barrett's oesophagus’. J Pathology. 200, 602-609 (2003).

[27] Z. Huang, A. McWilliams, H. Lui, D. I. McLean, S. Lam, H. Zeng, “Near-Infrared Raman Spectroscopy for optical diagnosis of lung cancer”, Int. J. Cancer: 107, 1047-1052 (2003).

[28] C. Murali Krishna, N. B. Prathima, R. Malini, B. M. Vadhiraja, R. A. Bhatt, D. J. Fernandes, P. Kushtagi, M. S. Vidyasagar, V. B. Kartha, “Raman spectroscopy studies for diagnosis of cancers in human uterine cervix”, Vib. Spectrosc. 41, 136-141, (2006).

[29] P. Crow, N. Stone, C. A. Kendall, J. S. Uff, J. A. M. Farmer, H. Barr and M. P. J. Wright, “The use of Raman spectroscopy to identify and grade prostatic adenocarcinoma in vitro”, Br. J. Cancer 89, 106-108, (2003).

[30] H. G. M. Edwards, A. G. Williams and B. W. Barry, “Potential Applications of FT-Raman Spectroscopy for Dermatological Diagnostics”, J. Mol Strut 347, 379-388 (1995).

[31] M. Gniadecka, P. Alshede Philipsen, S. Sigurdsson, S. Wessel, O. F. Nielsen, D. H. Christensen, J. Hercogova, K. Rossen, H. K. Thomsen, R. Gniadecki, L. K. Hansen, H. C. Wulf, “Melanoma Diagnosis by Raman Spectroscopy and Neural Networks: Structure Alterations in Proteins and Lipids in Intact Cancer Tissue”, J Invest Dermatol 122, 443-449(2004).

[32] M. Gniadecka, H. C. Wulf, N. Nymark Mortensen, O. F. Nielsen, and D. H. Christensen, “Diagnosis of basal cell carcinoma by Raman spectroscopy”, J. Raman Spectrosc. 28, 125-130 (1997.b).

[33] G. Shetty, C. Kendall, N. Shepherd, N. Stone and H. Barr, “Raman spectroscopy: elucidation of biochemical changes in carcinogenesis of oesophagus”, Br J Cancer 94, 1460-1464 (2006).

[34] M. J. Romeo, M. Diem, “Infrared spectral imaging of lymph nodes: Strategies for analysis and artifact reduction”, Vib. Spectrosc. 38 115-119 (2005).

[35] Krafft, C, Codrich D, Pelizzo G, Sergo V, “Raman mapping and FTIR imaging of lung tissue: congenital cystic adenomatoid malformation”, Analyst 133, 361-371 (2008).

[36] M. Mogensen, G. B. E. Jemec, “Diagnosis of Nonmelanoma Skin Cancer/Keratinocyte Carcinoma: A Review of Diagnostic Accuracy of Nonmelanoma Skin Cancer Diagnostic Tests and Technologies”, Dermatol Surg 33, 1158-1174 (2007).

[37] L. Brochez, E. Verhaeghe, E. Grosshans, E. Haneke, G. Piérard, D. Ruiter, J.-M. Naeyaert, “Inter-observer variation in the histopathological diagnosis of clinically suspicious pigmented skin lesions”, J Pathol. 196, 459-66 (2002).

[38] M. J. Trotter and A. K. Bruecks, “Interpretation of skin biopsies by general pathologists: diagnostic discrepancy rate measured by blinded review”. Arch. Pathol. Lab. Med. 127, 1489-1492 (2003).

1. A method of analysing a tissue sample comprising:
obtaining micro Raman spectra from one or more locations across the sample; and
applying a multivariate supervised statistical classification model to predict the class of each spectrum as dermis, epidermis or basal cell carcinoma (BCC).

2. The method of claim 1, further comprising creating a quantitative Raman spectroscopic image of the sample based on the classification from the model.

3. The method of claim 2, wherein said image represents any regions of dermis, epidermis and BCC that are present in the sample with different colours for each class of region.

4. The method of claim 1, wherein said step of applying a multivariate supervised statistical classification model comprises a linear discriminant analysis (LDA) model.

5. The method of claim 4, wherein an unsupervised k-means clustering is performed on the obtained micro Raman spectra, and then peak ratios of the centroids of each of those clusters are introduced as input to the LDA model and classified as dermis, epidermis or BCC.

6. The method of claim 4, wherein the LDA model is applied directly to the micro Raman spectra to classify each unknown spectra as dermis, epidermis or BCC.

7. The method of claim 1, wherein neighbouring spectra are binned and averaged together with the sample spectrum to reduce variability due to tissue heterogeneity.

8. The method of claim 1, wherein the model uses selected Raman bands chosen to reflect the biochemical differences between BCC and healthy skin regions.

9. The method of claim 8, wherein said selected Raman bands comprise vibrations specific to collagen 1, said bands from the sample being compared with the model to differentiate dermis from BCC.

10. The method of claim 9, wherein said vibrations specific to collagen 1 comprise vibrations corresponding to at least one of O—P—O phosphodiester and PO₂.

11. The method of claim 8, wherein said selected Raman bands comprise vibrations specific to DNA, said bands from the sample being compared with the model to differentiate epidermis from BCC.

12. The method of claim 11, wherein said vibrations specific to DNA comprise vibrations corresponding to at least one of C—C vibrations in protein backbones and Amide III vibrations in protein.

13. The method of claim 8, wherein said selected Raman bands are represented as the ratios of peak intensities at the selected Raman bands with the intensity of a reference band that shows low differences between dermis, epidermis and BCC classes.

14. The method of claim 13, wherein said reference band corresponds to the ring breathing of phenylalanine.

15. The method of claim 13, wherein said ratios are introduced as the input parameters in two consecutive linear discriminant analyses.

16. The method of claim 4, wherein:
a first linear discriminant analysis is performed to discriminate BCC and epidermis from dermis; and
a second linear discriminant analysis is performed to discriminate BCC from epidermis.

17. A multivariate supervised statistical classification model to predict the class of each spectrum as dermis, epidermis or basal cell carcinoma (BCC).

18. The model of claim 17, comprising data derived from the mean spectra of a plurality of tissue samples.

19. The model of claim 17, using selected Raman bands chosen to reflect the biochemical differences between BCC and healthy skin regions.

20. The model of claim 19, wherein said selected Raman bands comprise vibrations specific to collagen 1, said bands from the sample being compared with the model to differentiate dermis from BCC.

21. The model of claim 20, wherein said vibrations specific to collagen 1 comprise vibrations corresponding to at least one of O—P—O phosphodiester and PO₂.

22. The model of claim 19, wherein said selected Raman bands comprise vibrations specific to DNA, said bands from the sample being compared with the model to differentiate epidermis from BCC.

23. The model of claim 22, wherein said vibrations specific to DNA comprise vibrations corresponding to at least one of C—C vibrations in protein backbones and Amide III vibrations in protein.

24. The model of claim 19, wherein said selected Raman bands are represented as the ratios of peak intensities at the selected Raman bands with the intensity of a reference band that shows low differences between dermis, epidermis and BCC classes.

25. The model of claim 24, wherein said reference band corresponds to the ring breathing of phenylalanine.

26. Apparatus for analysing a tissue sample comprising:
a stage for receiving a tissue sample;
a Raman micro-spectrometer; and
a multivariate supervised statistical classification model arranged to be applied to the micro Raman spectra of the tissue sample obtained from the Raman micro-spectrometer, to predict the class of each spectrum as dermis, epidermis or basal cell carcinoma (BCC).

27. The apparatus of claim 26, comprising means to display a quantitative Raman spectroscopic image of the sample based on the classification from the model.

28. The apparatus of claim 27, wherein said image represents any regions of dermis, epidermis and BCC that are present in the sample with different colours for each class of region.

29. The apparatus of claim 26, wherein said multivariate supervised statistical classification model comprises a linear discriminant analysis (LDA) model.

30. The apparatus of claim 29, comprising means for performing an unsupervised k-means clustering on the obtained micro Raman spectra, and to introduce peak ratios of the centroids of each of those clusters as classified dermis, epidermis or BCC inputs to the LDA model.

31. The apparatus of claim 29, comprising means for applying the LDA model directly to the micro Raman spectra to classify each unknown spectra as dermis, epidermis or BCC.

32. The apparatus of claim 26, comprising means for binning and averaging together neighbouring spectra with the sample spectrum to reduce variability due to tissue heterogeneity.

33. The apparatus of claim 26, wherein said model is arranged to use selected Raman bands chosen to reflect the biochemical differences between BCC and healthy skin regions.

34. The apparatus of claim 33, wherein said selected Raman bands comprise vibrations specific to collagen 1, said bands from the sample being compared with the model to differentiate dermis from BCC.

35. The apparatus of claim 34, wherein said vibrations specific to collagen 1 comprise vibrations corresponding to at least one of O—P—O phosphodiester and PO₂.

36. The apparatus of claim 33, wherein said selected Raman bands comprise vibrations specific to DNA, said bands from the sample being compared with the model to differentiate epidermis from BCC.

37. The apparatus of claim 36, wherein said vibrations specific to DNA comprise vibrations corresponding to at least one of C—C vibrations in protein backbones and Amide III vibrations in protein.

38. The apparatus of claim 34, wherein said selected Raman bands are represented as the ratios of peak intensities at the selected Raman bands with the intensity of a reference band that shows low differences between dermis, epidermis and BCC classes.

39. The apparatus of claim 38, wherein said reference band corresponds to the ring breathing of phenylalanine.

40. The apparatus of claim 38, wherein said ratios are introduced as the input parameters in two consecutive linear discriminant analyses.

41. The apparatus of claim 26, wherein a first linear discriminant analysis is performed to discriminate BCC and epidermis from dermis; and a second linear discriminant analysis is performed to discriminate BCC from epidermis.

42. A computer program product carrying instructions for the performance of the method of claim 1.

43. A computer program product carrying instructions for the embodiment of the model of claim 17.

44. A computer program product carrying instructions for control of the apparatus of claim 26.

45. A method for the surgical removal of a BCC tumor, comprising:
removing a first tissue sample from a patient;
analysing the first tissue sample according to the method of claim 1; and
removing a subsequent tissue sample from the patient based on the or any detection of BCC in the first tissue sample.
